(19) United States
(12) Patent Application Publication
(10) Pub. No.: US 2003/0147944 A1
(43) Pub. Date: Aug. 7, 2003

(54) Lipid carrier compositions with protected surface reactive functions

(76) Inventors: Lawrence D Mayer, North Vancouver (CA); Gigi Chiu, Richmond (CA); Marcel B Bally, Bowen Island (CA)

Correspondence Address: Kate H Murashige, Morrison & Foerster, Suite 500, 3811 Valley Centre Drive, San Diego, CA, 92130-2332, US

(21) Appl. No.: 10/149,559

(22) PCT Filed: Dec. 11, 2000
(86) PCT No.: PCT/CA00/01494

(51) Int. Cl. A61K 9/127

(52) U.S. Cl. 424/450

(57) ABSTRACT

The liposomes of the invention have a reactive surface that demonstrates reduced interaction with macromolecules and increased blood circulation time. The reactive surface may comprise phosphatidylserine. The liposomes are protected by the presence of high levels of a hydrophilic polymer conjugated to a lipid. The invention further provides means for adjusting the appropriate ratio of hydrophilic polymer to a reactive lipid by a) determining the reactivity of the lipid; b) determining the time required for the carrier to reach its desired target location; c) determining the affinity of desired interactions with the reactive surface; and d) incorporating in the liposome or lipid carrier the amount of polyethylene glycol required to protect the reactive surface.

### TECHNICAL FIELD

The present invention is directed toward liposome and lipid-based therapeutic carrier systems.

### BACKGROUND OF THE INVENTION

The effectiveness of therapeutic agents for the treatment of many human diseases including cancer, autoimmune disease and cardiovascular disease are often limited and complete cures of these ailments are seldom obtained. The fact that very efficient biological activity can often be demonstrated with these agents in test tube or tissue culture assays suggests that the pharmacodistribution properties of these drugs after in vivo administration may play a major role in determining their therapeutic index.

Liposome and other lipid-based carrier systems have been extensively developed and analyzed for their ability to improve the therapeutic index of drugs by altering the pharmacokinetic and tissue distribution properties of the encapsulated or associated agents. This approach is aimed at using liposomes and lipid-based carriers to reduce exposure of healthy tissues to the therapeutic agents while increasing drug delivery to the disease site. In order to achieve this goal, liposomes must be stable upon exposure to the numerous protein, carbohydrate and lipid components after systemic administration to humans and animals. This has been accomplished by utilizing liposomes composed of neutral (no net charge) lipids such as phosphatidylcholine (PC) as well as cholesterol. Incorporating reactive charged lipids such as phosphatidylserine (PS) in liposomal compositions results in rapid recognition and clearance of the liposomes from the circulation, (Kirby, et al., (1980) Biochem J. 186(2):591-8) thus, reducing drug delivery to disease sites (Allen, et al. (1998) Proc. Natl. Acad. Sci. USA 85:8067-8071). In addition, attempts to improve the liposomal distribution by attaching molecules such as antibodies and other proteins to liposome surfaces has resulted in immune recognition and rapid clearance of the liposomes from the blood (Shek, et al., (1983) Immunology 50(1): 101-6; Aragnol, et al. (1986) Proc Natl Acad Sci USA 83(8):2699-703).

Grafting a hydrophilic polymer such as a polyalkylether to the surface of liposomes has been utilized to “sterically stabilize” the liposome thereby minimizing protein adsorption to liposomes. This results in enhanced blood stability and increased circulation time, reduced uptake into healthy tissues, and increased delivery to disease sites such as solid tumor (see: U.S. Pat. Nos. 5,013,556 and 5,593,622; and Patel, et al., (1992) Crit Rev Ther Drug Carrier Syst 9:39-90). Typically, the polymer is conjugated to a lipid component of the liposome. A preferred hydrophilic polymer is polyethylene glycol (PEG). Such a polymer conjugated lipid may be mixed with other lipids in preparation of liposomes or the conjugated lipid may be exchanged in the liposome from another source (such as from a vesicle or micelle containing the conjugated lipid). Alternatively, the polymer may be conjugated to a lipid component present on the exterior surface of a previously prepared liposome (see: U.S. Pat. No. 6,132,763). Typically, PEG is conjugated to lipids having a head group that contains a primary amine but other PEG-lipid derivatives are known. As well, the literature describes various moieties that may be situated between a lipid and a hydrophilic polymer. A commonly used conjugate is PEG derivatized to distearylphosphatidylethanolamine (DSPE) with the resulting conjugate being termed PEG-DSPE.

Literature dating from the beginning of the use of PEG lipids in liposomes suggests that the amount of the PEG lipid that may be used when preparing a liposome could range as high as 20 or 50 mol %, with the molecular weight of the PEG varying from as low as 50 to as high as 20,000 (see: U.S. Pat. Nos. 5,013,556 and 5,593,622). However, this literature did not correlate the amount or the size of the PEG polymer to changes in liposome behaviour or to the actual composition of the resulting liposome. Subsequently, it became known that the molecular weight of the PEG affects the extent to which a liposome is sterically stabilized as reflected, for example, by the circulation time of the liposome. Also, the amount of a PEG-lipid that will be incorporated into the liposome is affected by the molecular weight of the PEG.

It has been reported that PEG of about 2000 daltons (Da) is optimal for increasing the circulation time of a liposome while still making the liposome surface available for epitope recognition (Allen, T. A. (1994) Trends in Pharmacological Studies 15(7):215-220). Further, the literature shows that there are limits to the amount of PEG-conjugated lipids that can actually be incorporated into a liposome. This is because the PEG-lipid will form non-bilayer phases such as micelles and non-vesicle structures such as bilayer discs.

It has been reported that the level at which liposomes become saturated with a 1900 or 5000 molecular weight PEG-lipid conjugate is about 5-7 mol % (Allen, T. A., et al. (1991) Biochem. Biophys. Acta. 1062:142-8). This result conforms with the level reported by Edwards, K., et al. with respect to the point at which bilayer discs form (Biophysical Journal, 73:258-266 (1997)). This amount of PEG-lipid also corresponds with the upper limits of the most preferred amounts reported in early literature (see: U.S. Pat. No. 5,593,622) and corresponds to the amount of PEG (2000) that is generally perceived to be useful for sterically stabilizing liposomes for drug delivery purposes (see: Du, H., et al. (1997) Biochemica Biophysica Acta 1326:236-248; and, Bradley, A. J., et al. (1998) Archives of Biochemistry and Biophysics 357:185-194). While Bradley, A. J., et al. (1998) [supra] reported that it was possible to actually incorporate up to 15 mol % PEG-lipid into cholesterol based liposomes using an excess amount of the PEG-lipid in the source mixture, fully one-third of the PEG-lipid was lost immediately upon addition to serum.

Despite early sourced literature suggesting that PEG-conjugated liposomes may include PS as one of the components of the liposome (U.S. Pat. No. 5,593,622), it became known that the steric stabilization effects of PEG are lost when a reactive lipid such as PS is included in the liposome and that such liposomes are rapidly cleared from the blood after intravenous administration. Holland, J. W., et al. demonstrated that large unilamellar vesicles (LUV) could be made by mixing equimolar amounts of PS and phosphatidylethanolamine (PE) with from 2-10 mol % PEG (2000 or 5000 Da) conjugated lipid, with the resulting effect being inhibition of the LUVs ability to fuse (Biochemistry 1996, 35:2618-2624). While no study was done by Holland, et al. of bloodstream circulation times of such lipid compositions, Klibamov, A. L., et al. reported drastically reduced blood concentrations after 5 hours for liposomes containing a ratio of 0.15 PS to 3.3 total lipid (about 6.5 mol % PS) and an equal amount of PEG (5000) conjugated to phosphatidylethanolamine (PE) (Biochem. Biophys. Acta. 1991, 1062:142-8). Further, Allen, et al. (1991) [supra] demonstrated rapid clearance of liposomes made by mixing lipids with 10 mol % PS and with what was reported as being an amount of PEG (1900)-DSPE (10 mol %) in excess of the amount required to saturate a liposome. From these reports, it is apparent that reactive liposome surfaces, particularly PS containing liposomes, are not compatible with conventional steric stabilization approaches and will exhibit inferior characteristics for therapeutic applications in vivo.

Although liposomes displaying stability in the blood after systemic administration can increase the delivery of encapsulated or associated agents to certain disease sites, the therapeutic improvements are much smaller than the degree of enhanced disease tissue uptake. This is due to the fact that the stability properties allowing efficient transport of these lipid-based carriers through the body inhibit bioavailability of the encapsulated agent once at the disease site. Inclusion of reactive components such as PS into the membrane surface is useful to improve targeting (e.g. to the reticuloendothelial system), intracellular delivery and/or drug release at the disease site (e.g. a tumor), or to take advantage of a therapeutic effect (e.g. cytotoxicity) of the reactive component. Consequently, the ability to generate liposomes and other lipid-based carriers containing reactive surface components such as PS that are protected from non-specific interactions in the blood while being available for desired interactions at the disease site would be of value for therapeutic drug delivery applications in disease conditions such as cancer and inflammation.

### SUMMARY OF THE INVENTION

This invention is based on the discovery that liposomes containing a reactive phospholipid such as PS can be made to incorporate well over 10 mol % (relative to total lipid content) of a hydrophilic polymer conjugated lipid. Further, the hydrophilic polymer stabilizes the resulting liposomes, providing much enhanced longevity of the liposomes while in blood circulation. These results are contrary to the previous wisdom concerning reactive liposomes and incorporation of PEG-lipids into a liposome.

The present invention provides means for controlling the exposure of reactive liposome surfaces to molecules that interact with them. This is accomplished through the incorporation of elevated membrane concentrations of hydrophilic polymer conjugated lipids in the liposomes, beyond those previously used for drug delivery applications. The nature of molecular interactions with the reactive liposome surface can be controlled by manipulation of the hydrophilic polymer content of the liposome. The specific composition of such reactive surface containing liposomes or lipid carriers may be selected based on the concentration of the reactive species on the liposome surface, the size of the molecule with which the reactive species interacts and the affinity of the interaction between external molecule and reactive lipid species as well as the structure of the hydrophilic polymer attached to the membrane surface. This controlled exposure can result in complete inhibition of reactive surface interactions with macromolecules or partially attenuated interactions. Further, the liposomes may be designed to permit an increase in reactive surface interactions over time by employing known techniques for modulating the rate of PEG exchange from a liposome in the bloodstream.

This invention provides a lipid carrier for administration to a warm blooded animal comprising one or more reactive phospholipids and, wherein the carrier has an outer leaflet comprising one or more hydrophilic polymer-lipid conjugates in an amount equivalent to that provided if the lipid carrier is formed in the presence of greater than 10 mol % hydrophilic polymer-lipid conjugates relative to total lipid content of the carrier. This means that in cases where the one or more hydrophilic polymer-lipid conjugates are predominantly in the outer leaflet of the carrier, the density of hydrophilic polymer on the outer leaflet is that which is equivalent to having the total concentration of the one or more hydrophilic polymer-lipid conjugates in the entire carrier be greater than 10 mol %. The latter situation may exist when the carrier is preformed and a hydrophilic polymer-lipid conjugate is added to or formed in the outer leaflet of the carrier. This invention also provides the aforementioned carrier wherein the total concentration of the one or more hydrophilic polymer-lipid conjugates in the carrier relative to total lipid content of the carrier is greater than 10 mol %. The latter situation contemplates forming the carrier with greater than 10 mol % hydrophilic polymer-lipid conjugate in the lipids used to form the carrier. In either situation, the equivalent amount, or total concentration of the one or more hydrophilic polymer-lipid conjugates may be at least about 12 mol % or 15 mol %, with the proportion being selected according to type and polymer size and the desired amount of protection of the reactive surface. Preferably, the equivalent amount or total concentration of hydrophilic polymer-lipid conjugate in the carrier, as described above will not exceed about 20 mol %. The carrier will typically comprise one or more structural or bulk lipids and may also encapsulate a non-lipid therapeutic agent as described herein.

In one embodiment the hydrophilic polymer-lipid conjugate is a 500-5000 Dalton molecular weight PEG derivatized to a phosphatidylethanolamine. The reactive liposome surface contains reactive phospholipids, including phosphatidylserine (PS).

A desired biological behaviour of reactive surface-containing liposomes can be achieved through the method of this invention. In this method, liposomes or lipid carriers containing reactive surfaces are protected using an elevated hydrophilic polymer-lipid conjugate concentration. One or more PEG polymers of different molecular weight or one or more equivalent hydrophilic polymers or mixtures thereof may be employed. The resulting protection of reactive surface exposure can be modified to enhance desired interactions while reducing undesirable interactions with the liposomes or lipid carriers. This controlled exposure can be exploited for various therapeutic applications.

This invention also provides a method of obtaining an appropriate ratio of one or more hydrophilic polymer conjugated lipids to one or more reactive phospholipids for controlling liposome and lipid carrier reactivity comprising a) determining reactivity of a phospholipid, b) determining a time required for a liposome or lipid carrier to reach a target in an animal body, c) determining affinity of desired interactions with the reactive lipid while protected with a hydrophilic polymer-lipid conjugate and d) incorporating in the liposome or lipid carrier, the amount of hydrophilic polymer conjugated lipid required to protect the reactive surface until the desired interaction is to occur.

This invention also provides a method for preparing liposome or lipid carriers of this invention by combining from about 0.1% to about 50 mol % reactive phospholipids with one or more hydrophilic polymer-lipid conjugates and bulk or structural lipids. The reactive liposome surface may be further comprised of charged lipids, peptide or protein-derivatized lipids, surface adsorbed proteins, carbohydrates or nucleic acid polymers, carbohydrate-derivatized lipids or small molecule derivatized lipids.

This invention also provides a method for utilizing reactive surface-containing liposomes of this invention for the purpose of delivering therapeutic bioactive agents. Reactive surface-containing liposomes and lipid carriers as described above in the present invention can be loaded with bioactive agents utilizing methods known to those skilled in the art. For example, the reactive surface of the liposome or lipid carrier of this invention may contain bioactive lipids such as ceramides, or the liposomes or lipid carriers may contain a bioactive agent such as an anticancer agent encapsulated inside the aqueous compartment of the liposome. The reactive surface-containing liposomes or lipid carriers of this invention may contain a bioactive agent such as an antisense oligodeoxynucleotide associated covalently or non-covalently with the membrane.

Liposomes or lipid carriers of this invention may be combined with a pharmaceutical excipient or diluent.

This invention also provides a method including administering a liposome or lipid carrier of this invention to a warm-blooded animal for therapeutic purposes. For example, a therapeutically effective amount of an above-mentioned therapeutic agent may be administered intravenously. This method may comprise treating cancer or inducing thrombogenesis by administering reactive surface-containing liposomes or lipid carriers of this invention which have, or which will have over time in the bloodstream, a cytotoxic or thrombogenic effect. Also, liposomes of this invention may be used in vitro, for example as cytotoxic agents or in blood clotting assays. In the latter case, the liposomes may include a tissue factor such as is described in U.S. Pat. No. 5,314,695.

In another aspect of the present invention, the reactive surface-containing liposomes or lipid carriers of this invention are used to improve target specific binding and accumulation while minimizing destabilization, elimination or immune recognition of the liposomes or lipid carriers in warm blooded animals. The reactive surface-containing liposomes or lipid carriers may contain a surface associated targeting ligand such as an antibody or peptide ligand. The liposome may contain a bioactive agent such as an anticancer agent.

In another aspect of the invention, reactive surface-containing liposomes or lipid carriers are activated in a warm blooded animal by a chemical or physical trigger. Examples include those triggers known in the art for causing cleavage of hydrophilic polymers from a lipid surface. The trigger may interact with the reactive surface of the liposomes or lipid carriers under conditions where other undesirable interactions are inhibited through use of this invention.

In another aspect of the invention, reactive surface-containing liposomes or lipid carriers of this invention are used in therapeutic applications to induce thrombogenesis, to inhibit cancer cell proliferation, or for treatment of cancer. In one embodiment, the reactive surface-containing liposomes or lipid carriers will contain PS. The reactive surface-containing liposomes or lipid carrier may exhibit selective thrombogenic or cytotoxic activity.

### BRIEF DESCRIPTION OF THE FIGURES

FIG. 1: is a graph showing size exclusion chromatography of 10 mol % PS liposomes containing 15 mol % DSPE-PEG 2000, at stock concentrations on a Bio-Gel A-15 m column. The liposomes were labeled with traces of [¹⁴C]-CHE (open circles) and [³H]-DSPE-PEG 2000 (open squares).

FIG. 2: is a graph showing the effect of incorporating DSPE-PEG 2000 on the plasma elimination curves of PS liposomes. Three mice were used for each data point, and the error bars represent the standard errors. The plasma elimination curves for the following liposomes were determined and plotted: DSPC/Chol 55:45 (squares), DOPS/DSPC/Chol 10:45:45 (circles), DSPE-PEG 2000 incorporated at 5 mol % (up triangles), 10 mol % (down triangles) and 15 mol % (diamond) in DOPS 10%/DSPC 45%/Chol 45%, and DSPE-PEG 2000/DSPC/Chol 5:50:45 (open squares).

FIG. 3: are graphs showing rate of thrombin formation in the presence of various liposome formulations assayed by an in vitro chromogenic assay. Panel A relates to PS liposomes with various mole percentages of PS. Panel B relates to DOPS 10%/DSPC/Chol with various mole percentages of DSPE-PEG 2000 incorporated. Panel C relates to DOPS 10%/DSPC/Chol with various mole percentages of DSPE-PEG 750 incorporated. Liposome concentrations used were 75 μM. The minimum rate of thrombin formation that could be characterized by the assay system was 0.465 mol thrombin.min⁻¹.mol⁻¹Xa, and the asterisks represent rates that were below the minimum. The assay was done in triplicate and the error bars represent standard deviations.

FIG. 4: are graphs showing the use of (Panel A) DSPE-PEG 750 and (Panel B) DSPE-PEG 2000 to inhibit the clotting activity of PS liposomes. The % inhibition was calculated as follows: % inhibition=(t_{PEG}−t_{PS})/(t_{blank}−t_{PS})×100 where t represented the clotting time of each type of liposome as determined by the in vitro clotting time assay. Liposomes with 10 mol % (solid circles) and 20 mol % (open circles) of DOPS in DSPC/Chol were compared. The liposome concentrations used in Panel A and Panel B were 0.4 mM and 0.2 mM respectively. Data points were determined in triplicate, and the error bars represent standard deviations.

FIG. 5: are graphs comparing the viability of LCC6 breast cancer cells exposed to differing concentrations of liposomes having 7.5 mol % DPPE-PEG 2000 in the outer leaflet only, and containing 20% PS of varying acyl chain length. Panel A compares viable cells remaining 24 hours after addition of DMPC/PS liposomes in which the PS is of varying acyl chain length. Panel B compares viable cells remaining 24 hours after addition of DMPC/PS/DPPE-PEG 2000 liposomes in which the PS is of varying acyl chain length.

FIG. 6: are graphs showing inhibition of clotting activity by DOPS 10%/DSPC/Chol liposomes containing PEG 2000 conjugated to PE moieties having different chain lengths, following desorption of PEG 2000 lipid conjugates for various lengths of time (Panel B). Desorption was done in vitro by permitting exchange of the PEG-lipid conjugate to EPC/Chol (55:45) multilamellar vesicles (MLV). The liposomes were separated from the MLV by centrifugation. [³H]-PEG-lipid and [¹⁴C]-CHE were used as markers and radioactivity in the supernatant was assayed by liquid scentillation (Panel A).

### DETAILED DESCRIPTION OF THE INVENTION

Throughout this specification, the following abbreviations have the indicated meaning. PEG: polyethylene glycol; PEG preceded or followed by a number: the number is the molecular weight of PEG in Daltons; PEG-lipid: polyethylene glycol-lipid conjugate; PE-PEG: polyethylene glycol-derivatized phosphatidylethanolamine; MPS: mononuclear phagocytic system; PE: phosphatidylethanolamine; PS: phosphatidyl-serine; DOPS:1,2-dioleoyl-sn-glycero-3-[phospho-L-serine]; PC: phosphatidylcholine; EPC: egg phosphatidylcholine; SM: sphingomyelin; DSPC:1,2-distearoyl-sn-glycero-3-phosphocholine; DSPE-PEG 2000 (or 2000 PEG-DSPE or PEG₂₀₀₀-DSPE):1,2-distearoyl-sn-glycero-3-phosphoethanolamine-N-[polyethylene glycol 2000]; DSPE-PEG 750 (or 750PEG-DSPE or PEG₇₅₀-DSPE):1,2-distearoyl-sn-glycero-3-phosphoethanolamine-N-[polyethylene glycol 750]; DPPE-PEG2000: 1,2-dipalmaitoyl-sn-glycero-3-phosphoethanolamine-N-[polyethylene glycol 2000]; DMPE-PEG 2000: 1,2-dimyristolyl-sn-glycero-3-phosphoethanolamine-N-[polyethylene glycol 2000]; DMPC:1,2-dimyristolyl-sn-glycero-3-phosphatidylcholine; Chol: cholesterol; CH: cholesterylhexadecylether; POPE: 1-palmitoyl-2-oleoyl-sn-glycerol-3-phosphatidyl-ethanolamine.

The terms “lipid carrier” and “lipid carrier composition” in this specification include vesicles comprising one or more lipid bilayers. Such vesicles include unilamellar and multilamellar forms and liposomes. Liposomes are vesicles comprised of one or more concentrically ordered lipid bilayers encapsulating an aqueous phase. Formation of such vesicles (including liposomes) requires the presence of vesicle forming lipids. The bilayer surface of a liposome that is exposed to the exterior environment in which the liposome exists is termed herein “the outer leaflet”.

“Vesicle-forming lipid” as defined herein refers to an amphipathic lipid capable of assuming or being incorporated into a bilayer structure. This includes such lipids that are capable of forming a bilayer by itself or when in combination with another lipid or lipids. An amphipathic lipid is incorporated into a lipid bilayer by having its hydrophobic moiety in contact with the interior, hydrophobic region of the bilayer membrane and its polar head moiety oriented towards an outer, polar surface of the membrane. Most phospholipids belong to the former type of vesicle forming lipid whereas cholesterol is a representative of the latter type.

“Amphipathic lipid” refers to any lipid possessing a hydrophobic moiety which orients into a hydrophobic phase and a polar head moiety which orients towards the aqueous phase. Hydrophilicity arises from the presence of functional groups such as hydroxyl, phosphato, carboxyl, sulfato, amino or sulfhydryl groups. Hydrophobicity results from the presence of a long chain of aliphatic hydrocarbon groups.

Vesicle-forming lipids that may be incorporated into liposomes or lipid carriers of this invention may be selected from a variety of amphiphatic lipids, typically including phospholipids such as phosphatidylcholine (PC), phosphatidylethanolamine (PE), phosphatidic acid (PA), phosphatidylinositol (PI), or phosphatidylglycerol (PG); sterols such as cholesterol; and, sphingolipids such as sphingomyelin. In this specification, the terms “bulk” or “structural” with reference to lipids means a vesicle-forming lipid which contribute to structure of a lipid carrier or liposome but is not intended to include a “reactive phospholipid”.

“Hydrophilic polymer-lipid conjugate” refers to a vesicle-forming lipid covalently joined at its polar head moiety to a hydrophilic polymer, and is typically made from a lipid that has a reactive functional group at the polar head moiety in order to covalently attach to the hydrophilic polymer. Suitable reactive functional groups are for example an amino, hydroxyl, carboxyl, or formyl group.

The lipid in a hydrophilic polymer-lipid conjugate may be any lipid described in the art for use in such conjugates and is preferably a phospholipid such as phosphatidylcholine (PC), phosphatidylethanolamine (PE), phosphatidic acid (PA) or phosphatidylinositol (PI) having two acyl chains comprising between about 6 and 24 carbon atoms in length with varying degrees of unsaturation. However, sphyngolipids (such as sphyngomyelin), glycolipids (such as cerebrosides), gangliosides and ceramides may be used. While cholesterol may be used, it is not preferred due to the high rate at which cholesterol is lost from a lipid carrier when in the bloodstream. Most preferably the lipid in the hydrophilic polymer-lipid conjugate is a phosphatidylethanolamine including the dilauroyl, dioleoyl, dimyristoyl, distearoyl and dipalmitoyl forms.

The hydrophilic polymer used in this invention is a biocompatible polymer characterized by a solubility in water that permits the polymer chains to effectively extend away from the liposome surface out into the aqueous shell surrounding the liposome and a flexibility of the chains that produces a uniform surface coverage of the liposome. Preferably, the hydrophilic polymer is a polyalkylether. Suitable hydrophilic polymers include polyethylene glycol (PEG), polymethylethylene glycol, polyhydroxypropylene glycol, polypropylene glycol, polylactic acid, polyglycolic acid, polylactic/polyglycolic acid copolymers, or polyacrylic acid as well as those disclosed in U.S. Pat. Nos. 5,013,556 and 5,395,619. Preferably, the hydrophilic polymer has a molecular weight between about 500 and 5000 Daltons. Preferably, the polymer is PEG.

Methods to covalently attach polymers to a vesicle-forming lipid are well known in the art and generally involve activating chemical groups at a polymer end prior to reaction with a reactive functional group at the polar end of a vesicle-forming lipid (see for example U.S. Pat. No. 5, 395,619). Alternatively the reactive functional group at the polar end may be activated for reaction with the polymer, or the two groups may be joined through a concerted coupling reaction.

A hydrophilic polymer-lipid conjugate may be prepared to include a releasable lipid-polymer linkage such as a peptide, ester or disulfide linkage which can be cleaved under selective physiological conditions so as to expose a reactive liposome or lipid carrier surface once a desired biodistribution has been achieved, such as is disclosed in U.S. Pat. No. 6,043,094; or, in Kirpotin, D., et al. (1996) FEBS Letters, 388:115-188. Alternatively, the lipid in the conjugate, and in particular, its acyl chain length may be selected to provide for a desired rate of exchange of the polymers from a liposome to expose a reactive surface over time (see: Adlakha-Hutcheon, G., et al. (1999) Nature Biotechnology 17:775-779).

A hydrophilic polymer-lipid conjugate may also include a targeting ligand attached at the free end of the polymer to direct the liposome to specific cells. Derivatives of polyethyleneglycol that allow conjugation of a targeting ligand are for example, methoxy(hydrazido)polyethyleneglycol and bis(hydrazido)polyethyleneglycol.

Mixtures of hydrophilic polymer-lipid conjugates may be incorporated into a liposome or lipid carrier of this invention as an alternative means of controlling liposome and lipid carrier reactivity. This can be achieved either by mixing the different lipids in the preparation of the liposome or by incorporating a lipid grafted with two or more hydrophilic polymers. The latter method requires attachment of a bi- or multi-functional groups such as to the polar head of the lipid prior to coupling with individuals polymers.

In addition to the reactive phospholipid and hydrophilic polymer-lipid conjugate, the liposomes or lipid carriers of this invention may also contain therapeutic agents in their internal compartment or bound covalently or non-covalently to the lipid components. Additional lipids may make up the liposome or lipid carrier, including phospholipids such as phosphatidylcholine (PC), phosphatidylethanolamine (PE), phosphatidylglycerol (PG), phosphatidylinositol (PI), sphingolipids such as sphingomyelin (SM) and sterols such as cholesterol (Chol) may be included to provide necessary structural support as part of the structural or bulk lipid component.

“A reactive phospholipid” as defined herein is a phospholipid in which the polar head group terminates with an α-amino acid. The phosphate group is covalently joined at one end to the side chain of a the α-amino acid and at the other end to a three-carbon backbone connected to an hydrophobic portion through an ether, ester or amide linkage. Included in this class are the phosphoglycerides such as phosphatidylserine (PS) and the sphingolipids which have two hydrocarbon chains in the hydrophobic portion that are between 5-23 carbon atoms in length and have varying degrees of saturation. The amino acid may be natural or synthetic and of the D or L configurations. Preferably the side chain of the amino acid is a straight or branched alkyl group having between 1 and 3 carbons, including saturated, mono and disubstituted alkyls. The term hydrophobic portion, with reference to a reactive phospholipd refers to apolar groups such as long saturated or unsaturated aliphatic hydrocarbon chains. Preferably the reactive phospholipid is a phosphotriglyceride wherein the hydrophobic portion results from the esterification of two C₆-C₂₄ fatty acid chains with the hydroxyl groups at the 1- and 2-positions of glycerol. Most preferably the reactive phospholipid is a phosphatidylserine with the two fatty acid chains selected independently of each other from the group consisting of caproyl (6:0), octanoyl (8:0), capryl (10:0), lauroyl (12:0), mirystoyl (14:0), palmitoyl (16:0), stearoyl (18:0), arachidoyl (20:0), behenoyl (22:0), lingnoceroyl (24:0) and phytanoyl, including the unsaturated versions of these fatty acid chains in the cis or trans configurations such as oleoyl (18:1), linoleoyl (18:2), erucoyl (20:4) and docosahexaenoyl (22:6).

Determination of the amount of a component in a liposome or lipid carrier of this invention (e.g. a mol % value for a lipid component) may be carried out by any means known in the art. The determination may be made by measuring the amount of a component present in a liposome or lipid carrier or through knowledge of the amount of the component used when making the liposome or lipid carrier. Confirmation that the latter method conforms with the former method may be done, for example, by the method described in Example 1 below.

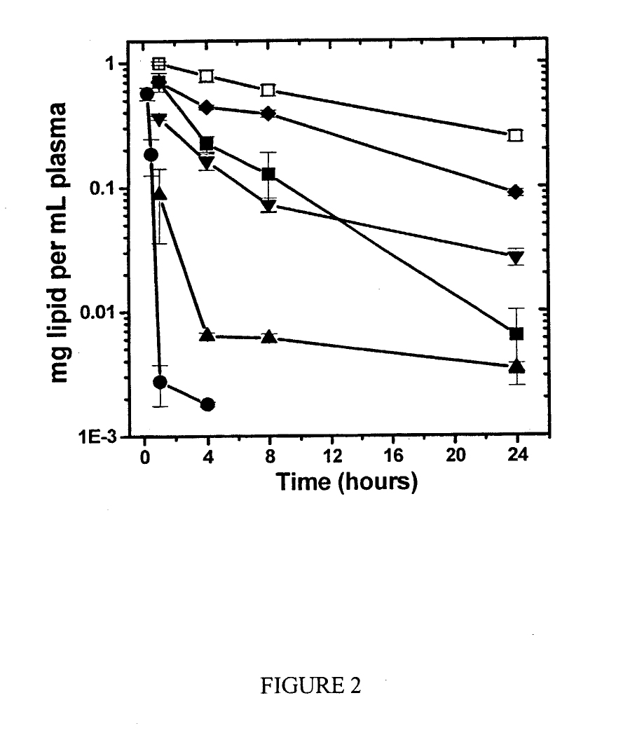

Lipid carriers or liposomes of this invention comprise greater than 10 mol % of a hydrophilic polymer-lipid conjugate as compared to the total lipid composition of the liposome or lipid carrier. This results in enhanced longevity (circulation time) while the liposome or lipid carrier of this invention is present in the bloodstream of a warm blooded animal. Preferably, a liposome or lipid carrier of this invention will be made such that the amount that would remain in the bloodstream of an animal 4 hours after intravenous administration is at least about 10 times (10 fold) the amount that which would remain 4 hours after intravenous administration of a reference liposome or lipid carrier. For purposes of this specification, a “reference” liposome or lipid carrier is one of similar composition to the liposome or lipid carrier of this invention to which the reference is compared, except that the reference contains no more than 5 mol % of a hydrophilic polymer-lipid conjugate. Preferably, the reference will consist of the same components as the liposome or lipid carrier of this invention and in the same relative proportions, taking into account the reduction of or absence of a hydrophilic polymer-lipid conjugate in the reference. Preferably, the amount of a liposome or lipid carrier remaining in the blood at 4 hours will be at least about 15; at least about 25, at least about 50 or at least about 75; and, even more preferably, at least about 100 times the amount of the reference liposome or lipid carrier that would remain at 4 hours. By selecting the amount of and molecular weight of the hydrophilic polymer-lipid conjugate employed in this invention, it is possible to have the amount remaining in the blood at 4 hours be as much as 300 times or more the amount that would remain of a reference containing no hydrophilic polymer-lipid conjugate (as is shown in Example 2 below and in FIG. 2). Further, the amount of a liposome or lipid carrier of this invention that would remain in the blood after 24 hours may be from about 5 to about 50 or more times the amount that would remain of a reference.

Determination of the amount of a liposome or lipid carrier that would remain in the bloodstream may be carried out by means known in the art, including the methods described in the Examples below involving intravenous administration to a test animal and monitoring of blood levels. This determination may be made for a liposome or lipid carrier intended for non-intravenous administration by formulating the liposome or lipid carrier in a suitable vehicle or diluent for intravenous administration, administering the formulation, and monitoring blood levels.

Compositions of the present invention may be generated by a variety of techniques including lipid film/hydration, reverse phase evaporation, detergent dialysis, freeze/thaw, homogenization, solvent dilution and extrusion procedures. The hydrophilic surface polymers may be incorporated as one of the lipid components at the time of initial liposome formation or may be added subsequent to liposome formation by incorporation through lipid exchange or derivatization of the liposome surface. The latter approaches can produce asymmetric surface polymer content in the bilayer membrane of liposomes whereas incorporation at the time of initial hydration will preferentially form liposomes with surface polymer equally distributed on both sides of the bilayer (Senior, et al. (1991) Biochem Biophys Acta 1062(1):77-82). Reactive phospholipids are typically added at the time of liposome formation whereas surface associated compounds (e.g. chemicals, drugs, peptides, proteins carbohydrates or polynucleotides) can be added during or after liposome formation. Bioactive agents (e.g. drugs) may be encapsulated inside liposomes of this invention by passive or active loading methodologies known in the art. Various combinations of surface reactive components, surface grafted steric stabilizing hydrophilic polymers, structural lipids such as phospholipids, sphingolipids and sterols, and bioactive agents can be used to adapt the surface reactivity, circulation stability, drug retention and disease site targeting of the liposomes and lipid carriers disclosed in this invention for specific applications such as drug delivery for cancer treatment. This can include formulations containing more than one bioactive agent and more than one surface reactive component.

The liposomes and lipid carriers of the present invention may be used in formulations that include peptides, proteins, carbohydrates or other small molecules either attached directly to the liposome surface or to a hydrophilic polymer for purposes of targeting the liposomes or lipid carriers to specific cell/tissue types. This application may also involve elevated PEG or other hydrophilic polymers to control the degree of exposure and binding affinity of targeting molecules. The degree of exposure of the targeting ligand and reactive surface components on liposomes and lipid carriers may be controlled together or independently by manipulating the concentration of the hydrophilic polymer, the density of reactive component on the liposome or lipid carrier surface and the concentration and position of the targeting ligand.

A method is provided for determining an appropriate ratio of reactive phospholipid to hydrophilic polymer-lipid conjugate to be used in making a liposome or lipid carrier of this invention that is suitable for use in a particular application. This method may be combined with incorporating the selected components into a liposome or lipid carrier. The method may include administrating the liposome or lipid carrier made by use of this method and may include activating the liposome or lipid carrier by a triggering event which may be exerted in the animal. This method includes determining the reactivity of a phospholipid (such as by the method described in Example 3) and determining a time required for a liposome or lipid carrier to reach a target location in an animal body. The time required to reach a location may include time required for uptake of a liposome or lipid carrier by target cells or tissue. The latter determination may be done experimentally or by calculation using known parameters in the body, cells, or tissue. Next, an affinity of a desired interaction at the target location for the liposome or lipid carrier is determined. Examples of this determination are in Examples 2 and 3 below in which a determination is made of the effect of varying levels of a hydrophilic polymer-lipid conjugate has on the affinity of a reactive lipid for a desired interaction at a target cell type or for a target blood protein or complex. Next, the liposome is made using amounts of reactive phospholipid and hydrophilic polymer-lipid conjugate selected to provide protection of the liposome or lipid carrier from clearance from the bloodstream for at least the time required to reach the target and to permit the desired interaction to occur at the target.

The concentration of hydrophilic polymer on a surface of liposomes or lipid carriers of this invention may be selected to provide controlled exposure of a reactive component in the membrane that can selectively interact with a triggering ligand in order to activate the liposome at the desired site. In this manner, biocompatibility of the liposomes or lipid carriers will be maintained while enabling a desired interaction between a specific reactive surface component and an intended secondary triggering ligand. This control of exposure may also utilize surface grafted hydrophilic polymers that are intended to be released from the liposome surface in time delayed or triggered mechanisms. These mechanisms may be based on the natural exchange of hydrophilic polymers out of the liposome or lipid carrier systems that takes place within a warm blooded animal (Adlankha-Hutcheon, G., et al. [supra]; Silvius, et al. (1993) Biochemistry 32:13318-26; and, Silvius, et al (1993) Biochemistry 32:3153-61), or on cleavage of the hydrophilic polymer from the liposome or lipid carrier surface (Kirpotin, et al. (1996) [supra]). Such controlled loss of hydrophilic polymer from the liposome surface can be used to increase the exposure of reactive components on the surface of the liposome or lipid carriers at a delayed time when improved disease site selectivity or targeting is achieved. These approaches may be applied to liposomes or lipid carriers of this invention to induce drug release at the desired site and time, to induce exposure of additional reactive components on the liposome or lipid carrier surface, to induce fusion of the liposome or lipid carrier with other membranes or cells or to induce recognition of the liposomes or lipid carriers by specific cells of the immune system.

Liposome and lipid carrier compositions of the present invention may be administered to warm-blooded animals, including humans. These liposome and lipid carrier compositions may be used to treat a variety of diseases in warm-blooded animals, the application of which depending on the particular bioactive agent or combination of agents and reactive surfaces incorporated in the liposome or lipid carrier formulation. Examples of medical uses of the compositions of the present invention include but are not limited to treating cancer, treating cardiovascular diseases such as hypertension, cardiac arrhythmia and restenosis, treating bacterial, fungal or parasitic infections, treating and/or preventing diseases through the use of the compositions of the present inventions as vaccines, treating inflammation or treating autoimmune diseases. For treatment of human ailments, a qualified physician will determine how the compositions of the present invention should be utilized with respect to dose, schedule and route of administration using established protocols. Such applications may also utilize dose escalation should bioactive agents encapsulated in liposomes and lipid carriers of the present invention exhibit reduced toxicity to healthy tissues of the subject.

For medical applications, formulations of the liposomes and lipid carriers of the present invention for parenteral administration are preferably in a sterile aqueous solution optimally comprised of excipients known to be tolerated by warm-blooded animals. For oral or topical applications, the liposome and lipid carrier compositions of the present invention may be incorporated in vehicles commonly used for the respective applications such as but not limited to creams, salves, ointments and slow release patches for topical medical applications and tablets, capsules, powders, suspensions, solutions and elixirs for oral applications.

The liposomes and lipid carrier compositions of the present invention may also be used for diagnostic purposes where the controlled exposure of reactive surfaces can provided improved selectivity of liposomes and lipid carriers with specific binding interactions while preventing unwanted interactions. Diagnostic applications may include administration of liposomes and lipid carrier compositions of the present invention as a parenteral agent to warm-blooded animals to detect the presence of specific disease sites or markers of disease. Such compositions may contain imaging agents including, but not limited to, radionuclides, magnetic resonance contrast agents and heavy atom contrast agents. Alternatively, diagnostic applications may utilize the liposomes and lipid carriers of the present invention for ex vivo diagnostic applications where selected interactions between proteins, carbohydrates or DNA and reactive surface containing liposomes can be used to detect the presence of these molecules while preventing unwanted complicating interactions with other molecules in solution.

### EXAMPLES

### Source of Materials

All lipids were obtained from Avanti Polar Lipids, except for those listed below.

DSPC and [³H]-DSPE-PEG 2000 were obtained from Northern Lipids (Vancouver, BC). [³H]- and [¹⁴C]-CHE were from NEN/Dupont. Cholesterol, ellagic acid, Sepharaose CL-4B were from Sigma. All blood coagulation proteins were from ICN (Aurora, Ohio). Thrombin chromogenic substrate S-2238 were from Chromogenix (Molndal, Sweden). Bio-Gel A-15 m and A-5 m size exclusion gel and gel filtration standards were from Bio-Rad (Mississauga, ON). An Oregon Green 514 protein labeling kit was from Molecular Probes (Eugene, Oreg.).

### Preparation of Large Unilamellar Liposomes

Lipids were prepared in chloroform solution and subsequently dried under a stream of nitrogen gas. The resulting lipid film was placed under high vacuum for a minimum of 2 h. The lipid film was hydrated in Hepes 20 mM/NaCl 150 mM buffer (pH 7.5) at 65° C. to form multilamellar vesicles. The resulting preparation was frozen and thawed five times prior to extrusion 10 times through two stacked 0.1 μm polycarbonate filters (Poretics Co., Canada) with an extrusion apparatus (Lipex Biomembranes, Vancouver, BC). The extrusion temperature was kept at 65° C. The size of the liposomes was determined by quasi-elastic light scattering using a Nicomp 370 submicron particle sizer operating at a wavelength of 632.8 nm. Incorporation and retention of DSPE-PEG 2000 in liposomes after preparation and subsequent in vivo administration were determined by size exclusion chromatography. Liposomes with traces of [¹⁴C]-CHE (as a general lipid marker) and [³H]-DSPE-PEG 2000 (as a PEG-lipid marker) were applied to a 42 cm×1.3 cm Bio-Gel A-15 m column (50-100 mesh) at various concentrations, and were eluted with Hepes 20 mM/NaCl 150 mM buffer (pH 7.5) at a flow rate of 0.5 mL/min regulated by a peristaltic pump. Aliquots from the 1-mL column fractions were counted directly in 5.0 mL scintillation fluid.

### Plasma Pharmacokinetics and Tissue Distribution of Liposomes

Lipsomes, labeled with [³H]-CHE as a non-exchangeable, non-metabolizeable lipid marker, were injected via lateral tail vein with a lipid dose of 50 mg/kg and an injection volume of 200 μL into ˜22 g female CD-1 mice. At various times, three mice from each group were terminated by CO₂ asphyxiation. Blood was collected by cardiac puncture, and was placed into EDTA-coated or heparin-coated microtainer collection tubes (Becton-Dickinson). After centrifuging the blood samples at 4° C. for 15 minutes at 1000 g plasma was isolated and visually showed no hemolysis. Aliquots of the plasma obtained were counted directly in 5.0 mL scintillation fluid. Liver, spleen and lungs were harvested from each group of mice to determine the biodistribution of liposomes. 0.5 mL Solvable (Packard BioScience Co.) was added to whole organs (spleen and lungs) or tissue homogenate (liver), and the mixture was incubated at 50° C. overnight. After cooling to room temperature, 50 μL EDTA 200 mM, 200 μL hydrogen peroxide 30%, and 25 μL HCl 10 N were added, and the mixture was incubated for one hour at room temperature. The mixture was added with 5.0 mL scintillation fluid and counted 24 hours later.

### Prothrombin Binding to Liposomes

Bovine prothrombin was labeled with the fluorescent dye Oregon Green 514, containing a reactive succinimidyl ester moiety that reacts with primary amines of the protein to form dye-protein conjugates. The fluorescently labeled prothrombin was incubated with various liposome composition at lipid concentrations of 0.2 and 0.4 mg/mL in the presence of 2.0 mM Ca²⁺ at 37° C. for 15 minutes. The mixture was then separated using Microcon 100 ultrafiltration devices by centrifugation at 3000g for 15 minutes. The filtrate, containing free protein, was measured for fluorescence with excitation and emission wavelengths set at 506 and 526 nm, respectively. The amount of prothrombin bound to liposomes was determined using a calibration curve constructed with known amounts of fluorescent labeled prothrombin and correcting for protein recovery using liposome-free prothrombin solutions.

### In vitro Chromozenic Assay for Factor Xa Activity

Formation of catalytically active prothrombinase protein complexes (factor Xa and factor Va) on liposome surfaces was determined employing a chromogenic substrate that is cleaved by enzymatically active thrombin and was described by Connor, J., et al. (1989) Proc. Natl. Acad. Sci. USA 86:3184-88. The “prothrombinase complex cocktail” contained the components for prothrombinase conversation under the following conditions: 8.0 nM (0.2 unit) factor Xa, 0.2 nM factor Va, 6 mM CaCl₂, and liposomes at various concentrations. These mixtures were incubated in Tris 50 mM/NaCl 120 mM buffer (pH 7.8) for five minutes at 37° C. Prothrombin (1 mM) was added to the cocktail, and the final mixture (150 μL) was incubated for three minutes. The conversion of prothrombin to thrombin was stopped by the addition of EDTA (15 mM final concentration). S-2238, which is a specific chromogenic substrate of thrombin, was added at 0.4 mM, and the rate of chromogen formation was monitored at 405 nm with a plate reader equipped with kinetic analysis software (Dynex Technologies Inc., Chantilly, Va.). A calibration curve was obtained under the same conditions with known amounts of thrombin, and the amount of thrombin formed in the assay were determined from the calibration curve.

### In vitro Clotting Time Assay

This assay was based on the activated partial thromboplastin time. An ellagic acid solution was used freshly prepared and diluted in 20 mM Hepes/150 mM NaCl. Human citrated plasma (50 μL) was pre-incubated with 10⁻⁵ M ellagic acid (50 μL) and liposomes (50 μL) for two minutes at 37° C. Calcium was then added to initiate the clotting reaction. The time at which the mixture changed from a liquid to a viscous gel was recorded, and was noted as the time for the clotting reaction to be completed.

### Example 1

### Incorporation of Elevated PEG Concentrations on Liposome Surfaces

Reactive phospholipid containing liposomes containing various amounts of PEG-lipid liposomes were made according to the above-described method by including PS (in the DOPS form) and DSPE derivatized with PEG in the mixture used to form the liposomes. High levels of different molecular weight PEG-lipids appeared to be incorporated into the PS containing liposomes.

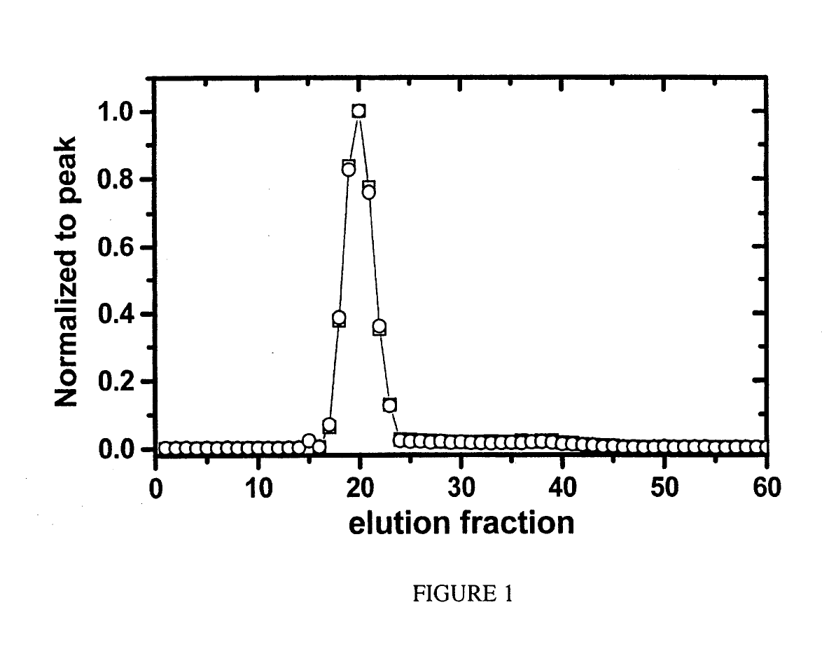

The previous literature suggested that an excess of PEG-lipid will not be incorporated with liposomes, but will form non-bilayer phases such as micelles; Therefore, the incorporation of 15 mol % DSPE-PEG 2000 into 10 mol % PS liposomes was examined with size exclusion chromatography to separate liposomes from DSPE-PEG 2000 micelles. The Bio-Gel A-15 m gel filtration column was first calibrated to resolve the liposomes from DSPE-PEG 2000 micelles by applying liposomes and DSPE-PEG 2000 micelles to the column immediately after mixing. As shown in FIG. 1, no micelle peak was observed in the elution profile of the stock preparation (54 mM) of 10 mol % PS liposomes containing 15 mol % DSPE-PEG 2000. Identical elution profiles were obtained when the stock liposomes were diluted to the concentrations used in in vitro (0.2 mM) and in vivo (6.2 mM) experiments. Based on the ratio of the radiolabeled DSPE-PEG 2000 and liposome marker ([¹⁴C]-CHE), the amount of DSPE-PEG 2000 present in PS liposome containing fractions reflected a DSPE-PEG 2000 composition of 14 mol % for all of the liposome concentrations. This data shows that elevated PEG-lipid content can be incorporated into reactive phospholipids containing liposomes in accordance with this invention, and that alternate lipid phases do not appear to be formed during the preparation or the dilution of such liposomes.

### Example 2

### Increased Biocompatibility and Circulation Longevity of PS Containing Liposomes with Elevated Membrane PEG Concentrations

Previous studies have demonstrated that PS liposomes are rapidly eliminated from circulation due to extensive binding of plasma proteins and subsequent uptake by the MPS. The effect of PEG-lipids on the plasma pharmacokinetics of 10 mol % PS liposomes made as described above was investigated. The percentage of initial dose remaining in circulation 4-hour and 24-hour post-injection and the AUC₀₋₂₄ₕ (“area under the curve” from 0-24 hours) for the various liposomes are summarized in Table 1. The results are also displayed in FIG. 2 which shows that conventional neutral DSPC/Chol liposomes exhibit a monophasic plasma elimination curve. However, inclusion of 10 mol % PS in DSPC/Chol liposomes changed the plasma elimination curve to biphasic, indicating rapid MPS uptake of the PS liposomes. The circulation longevity of DSPC/Chol liposomes was dramatically decreased with the inclusion of greater than 10 mol % PS, as reflected by the concentration of liposomes remaining in the circulation relative to the initial dose four and twenty four hours post-injection.

Among the various liposomes containing PS and PEG-lipid, those with 15 mol % DSPE-PEG 2000 exhibited a monophasic plasma elimination curve similar to that of the sterically stabilized DSPE-PEG 5%/DSPC/Chol liposomes (FIG. 2). The inclusion of 15 mol % DSPE-PEG 2000 in 10 mol % PS liposomes greatly increased the circulation longevity of the PS liposomes, giving 34% and 9.5% of initial dose remaining 4- and 24-hour post-injection, respectively. The AUC₀₋₂₄ₕ was also increased from 0.65 to 7.97 mg.mL⁻¹.h with the inclusion of 15 mol % DSPE-PEG 2000 in 10 mol % PS liposomes, reflecting a 12-fold increase in the AUC₀₋₂₄ₕ. The PS liposomes with 5 mol % DSPE-PEG 2000, although containing the same amount of PEG-lipid as the sterically stabilized neutral liposomes, were eliminated from the bloodstream more rapidly than DSPC/Chol liposomes, indicating the ineffectiveness of 5 mol % DSPE-PEG 2000 to protect PS containing liposomes.

TABLE 1
Plasma pharmacokinetics of various liposomes

| | % dose | | AUC_{0-24 h} |
|---|---|---|---|
| | remaining | | (mg.mL⁻¹ |
| Liposomes | 4 h | 24 h | .h)¹ |
| DSPC/Chol 55:45 | 22 ± 9 | 0.7 ± 0.1 | 4.02 |
| DOPS/DSPC/Chol 10:45:45 | 0.13 ± 0.01 | 0.08 ± 0.01 | 0.65 |
| DSPE-PEG 750/DOPS/DSPC/ | 2.9 ± 0.7 | 0.45 ± 0.08 | 1.30 |
| Chol 20:10:25:45 | | | |
| DSPE-PEG 2000/DOPS/DSPC/ | 34 ± 4 | 9.5 ± 0.7 | 7.97 |
| Chol 15:10:30:45 | | | |
| DSPE-PEG 2000/DSPC/Chol | 56 ± 10 | 18 ± 3 | 13.31 |
| 5:50:45 | | | |

¹AUC_{0-24 h} was calculated based on trapezoidal method.
²% dose remaining was an average ± S.D. of three to six mice. The injected lipid dose was 50 mg/kg.

### Example 3

### Controlled Exposure of PS Liposomes to PS-Binding Blood Coagulation Proteins With Elevated Membrane PEG Concentrations

The reactive phospholipid PS is known to be active in promoting blood clotting and has been shown to bind prothrombin. Calcium-dependent prothrombin binding to 10 mol % PS liposomes made according to the preceding method, was determined using fluorescently labeled protein and separating free and liposome bound pools under equilibrium conditions with ultrafiltration devices. Fluorescent derivatization did not significantly alter the binding properties of prothrombin to DSPC/Chol liposomes containing 10 mol % PS as free vs. bound protein fractions were similar to those for prothrombin binding to PS-containing liposomes reported previously using light scattering techniques. Negligible prothrombin association with liposomes was observed in the absence of PS under conditions where between 25% and 40% of the protein in solution was bound to 10 mol % PS liposomes (0.25:1 and 0.1:1 protein to lipid w/w ratios, respectively). Incorporation of PEG₂₀₀₀-DSPE at 5 mol % in DSPC/Chol (50:45 molar ratio) liposomes resulted in 28% and 37% inhibition of prothrombin binding at protein/lipid wt. ratios of 0.25:1 and 0.1:1, respectively. Increasing the amount of PEG₂₀₀₀-DSPE to 10% and 15% enhanced the inhibition of prothrombin binding to the 10% PS liposomes where an 85% decrease in protein binding was observed using 15% PEG₂₀₀₀-DSPE at a protein to lipid wt. ratio of 0.1:1 and a 75% protein binding decrease at the 0.25:1 protein to lipid ratio.

### Effects of PEG-Lipids on the Functional Activity of Membrane Bound Blood Coagulation Proteins

The results above demonstrate that elevated PEG-lipid content will reduce prothrombin binding to PS liposomes. The effect of PEG-lipids on the functional activity of membrane bound blood coagulation proteins was also examined.

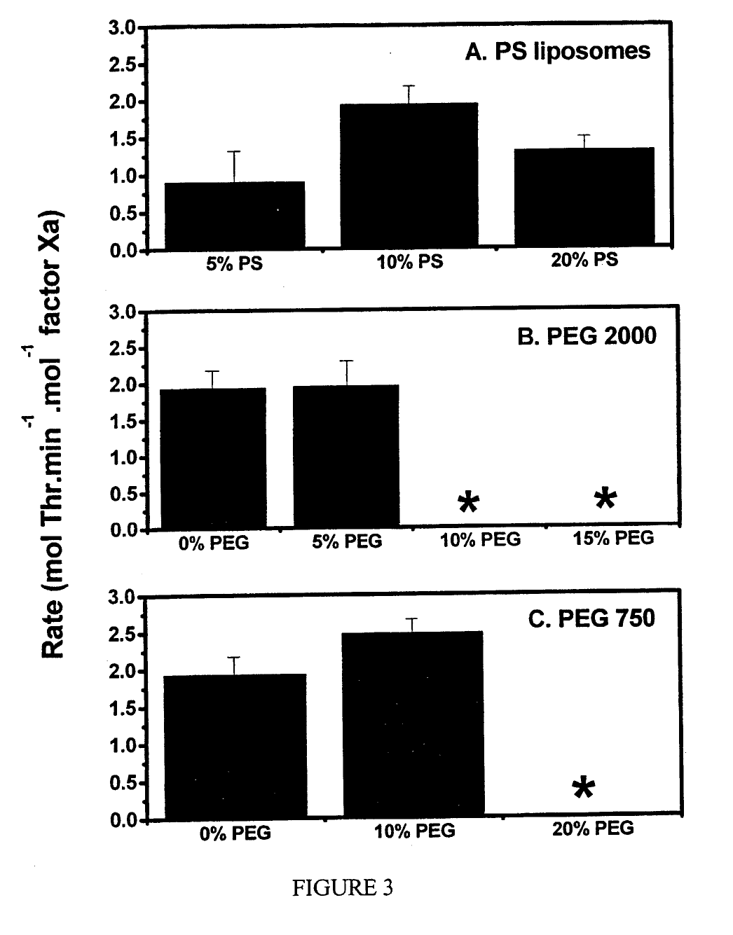

First, the effect of PEG-lipids on the catalytic activity of the prothrombinase complex was considered. The complex consists of factors Xa and Va assembled on negatively charged membrane surfaces and is responsible for the proteolytic activation of prothrombin to thrombin. The rate of thrombin formation by the prothrombinase complex in the presence of liposomes was monitored using a chromogenic substrate that is activated by thrombin as in the above-described assay. In the absence of PS in DSPC/Chol. liposomes, the rate of thrombin formation was negligible, and no substrate activation over mixtures devoid of liposomes was observed. Incorporating 10 mol % PS into DSPC/Chol liposomes resulted in a rate of thrombin formation of 1.94 mol thrombin.min⁻¹ mol⁻¹ factor Xa, which was the highest among the various PS containing liposomes tested (FIG. 3).

10 mol % PS liposomes were then used to evaluate the effectiveness of DSPE-PEG 750 and DSPE-PEG 2000 in inhibiting the assembly and the catalytic activity of the prothrombinase complex on the PS membrane surface. With 10 mol % DSPE-PEG 750 or 5 mol % DSPE-PEG 2000 in the PS liposomes, the rates of thrombin formation were 2.48 and 1.96 mol thrombin.min⁻¹mol⁻¹ factor Xa respectively, which were similar to those for the PS liposomes without PEG-lipid. Only by elevating the PEG-lipid content to 20 mol % for DSPE-PEG 750 or 10-15 mol % for DSPE-PEG 2000 could the rate of thrombin formation be reduced to <0.465 mol thrombin.min⁻¹.mol⁻¹ factor Xa.

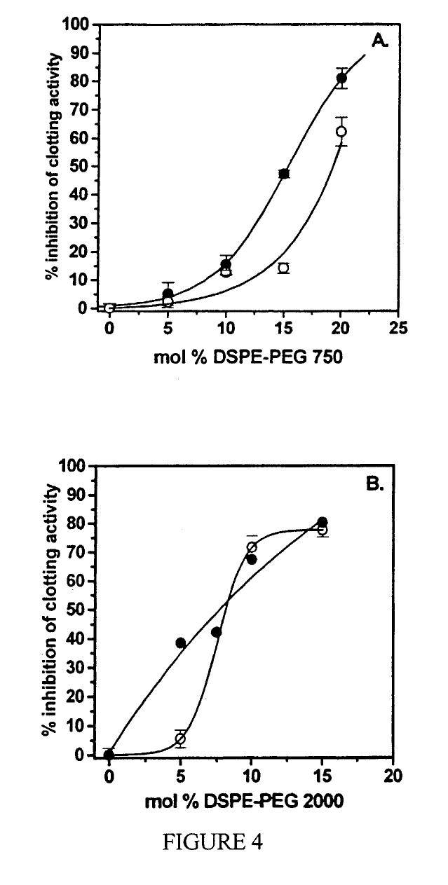

In addition to the prothrombinase complex, a PS membrane surface is involved in the proteolytic activation of several blood coagulation proteins and propagation of the blood coagulation cascade. Further, full clot formation requires the release of thrombin from the prothrombinase complex which can then enzymatically convert fibrinogen to fibrin. The impact of PEG-lipids on the comprehensive procoagulant activity of PS liposomes was determined using a modified activated partial thromboplastin time where exogenously added liposomes provided the catalytic membrane surface. The percent inhibition of clotting activity of 10 and 20 mol % PS liposomes by the PEG-lipids is presented in FIG. 4. When incorporated at ≦10 mol %, DSPE-PEG 750 inhibited approximately 15% of the clotting activity for 10 and 20 mol % PS liposomes. The inhibitory effect of DSPE-PEG 750 was sigmoidal, as reflected by the increase in the percent inhibition of the clotting activity for 10 and 20 mol % PS liposomes. When the level of DSPE-PEG 750 in PS liposomes was increased to 20 mol %, the inhibition of procoagulant activity was increased to 85% and 65% for 10 and 20 mol % PS liposomes, respectively. A similar result occurred with DSPE-PEG 2000 where low levels of the PEG-lipid in the PS liposomes provided modest inhibition to the clotting activity. Specifically, when DSPE-PEG 2000 incorporated at 5 mol %, 40% and 8% of the clotting activity was inhibited for 10 and 20 mol % PS liposomes, respectively. This inhibitory effect was increased to approximately 80% when the level of DSPE-PEG 2000 was increased to 15 mol % in the two PS liposomes.

These results show that liposomes of the present invention may be made to retain some coagulant activity or to be very inhibited in coagulant activity. Further, the size and amount of PEG may be selected to produce liposomes within the scope of this invention which will significantly increase in coagulant activity after PEG is lost from the liposome either due to the exchange processes that occur in the bloodstream or as a result of a triggering event that releases PEG from the liposome. In this way, thrombogenic liposomes of this invention can be targeted to a body location without the liposomes being first cleared from the bloodstream.

### Example 4

### Cytotoxicity of PS Containing Liposomes

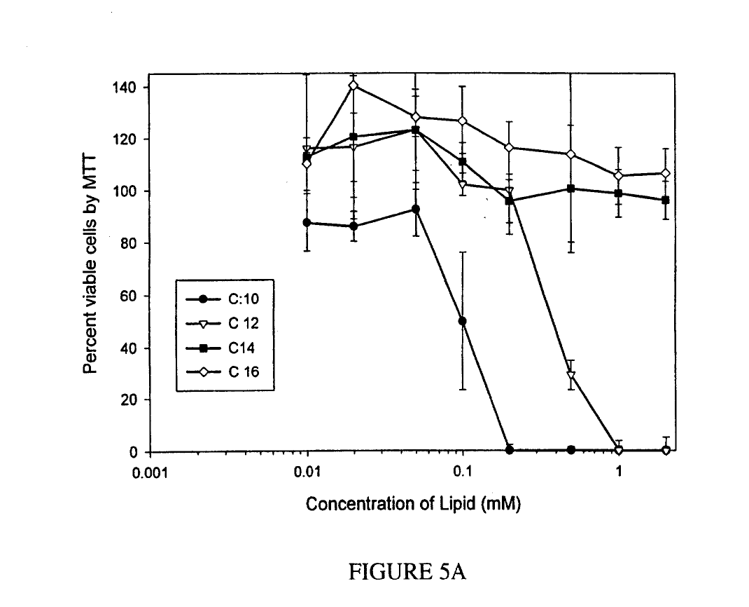

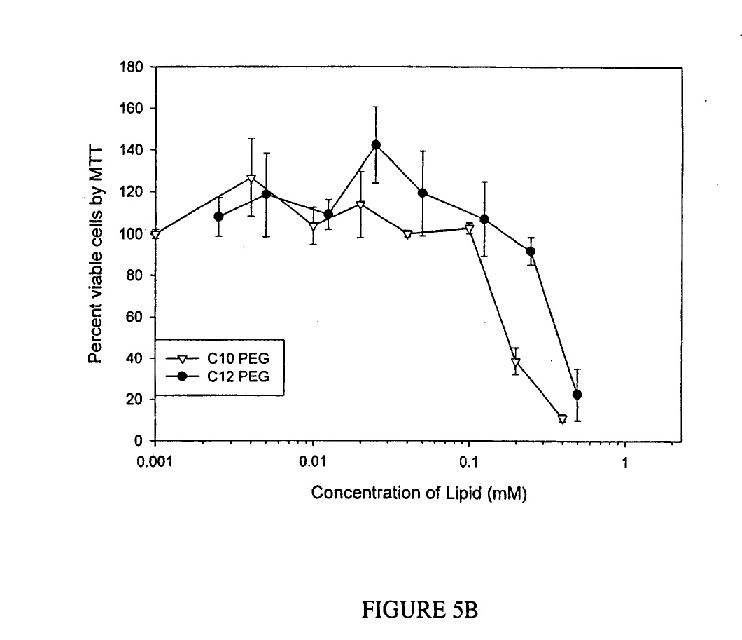

Liposomes were prepared with 20 mol % PS of varying acyl chain composition. The acyl chain lengths were chosen to be shorter, (C:10) and (C:12), equal (C:14). or longer (C:16) in length to the base lipid, DMPC. 7.5 mol % DPPE-PEG was exchanged from PEG micelles into an aliquot of each of the DMPC/PS liposomes by incubating liposomes and micelles at 37° C. overnight. The PEG was shown to be completely exchanged by size exclusion chromatography on Sepharose C1-4B columns. This results in the outer leaflet having a density of PEG equivalent to a liposome having a total PEG-lipid concentration relative to total lipid content of greater than 10 mol % (equivalent to the liposome having a total PEG-lipid concentration of about 15 mol %). PS liposomes were diluted in tissue culture media (DMEM 10% FBS) and added to LCC6, human breast cancer cells. The cells were incubated with the liposomes overnight, then viability was assessed with a MTT assay. The results are shown in FIG. 5.

These results show that liposomes containing PS are toxic to cells and in particular, those containing short (e.g. C:10 or C:12) acyl chains. Incorporation of a PEG-lipid into the liposome does not appear to significantly effect cytotoxicity. Therefore, liposomes of this invention may be used to inhibit proliferation or to kill cancer cells with the liposomes being protected from blood clearance by the hydrophilic polymer.

### Example 5

### Effect of Acyl Chain Length on Desorption of PEG-lipids

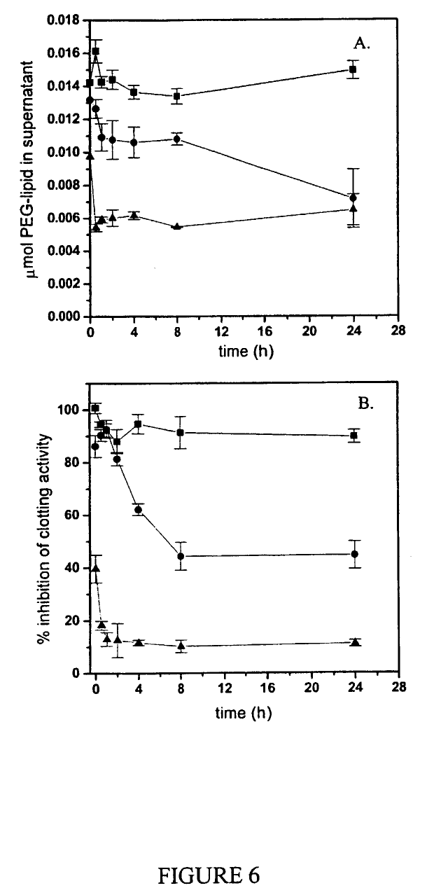

The effect of different acyl chain lengths of PE conjugated to PEG 2000 on the rate and the amount by which PS containing liposomes may be de-protected and clotting activity restored was investigated. The results are shown in FIG. 6. The clotting assay was the same as described above. Simulating loss of PEG-lipid conjugate from the liposome by exchange in vitro to other vesicles, shows that the amount by which PS containing liposomes become de-protected increases with a decreasing acyl chain length (from C:18-C:14). Inhibition of clotting activity was reduced the most with DMPE-PEG 2000, followed by DPPE-PEG 2000, and then by DSPE-PEG 2000.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

We claim:

1. A lipid carrier for administration to a warm blooded animal comprising one or more reactive phospholipids and, wherein the carrier has an outer leaflet comprising one or more hydrophilic polymer-lipid conjugates in an amount equivalent to that provided if the lipid carrier is formed in the presence of greater than 10 mol % of the hydrophilic polymer-lipid conjugates relative to total lipid content of the carrier.

2. The lipid carrier of claim 1 wherein the carrier is a liposome.

3. The lipid carrier of claim 1 or 2, comprising from about 0.1 to about 50 mol % relative to total lipid content of the carrier of one or more reactive phospholipids.

4. The lipid carrier of any one of claims 1-3, wherein a reactive phospholipid present in the carrier is a phosphatidylserine (PS).

5. The lipid carrier of claim 4 wherein the carrier comprises from about 10 to about 50 mol % PS.

6. The lipid carrier of claim 4 wherein the carrier comprises from about 10 to about 30 mol % PS.

7. The lipid carrier of any one of claims 1-6, wherein total concentration of the one or more hydrophilic polymer-lipid conjugates in the carrier relative to total lipid content of the carrier is greater than 10 mol %.

8. The lipid carrier of any one of claims 1-7, wherein a hydrophilic polymer-lipid conjugate present in the carrier is a lipid conjugated to a polyalkylether of from about 500 to about 5000 Daltons.

9. The lipid carrier of claim 8 wherein the polyalkylether is polyethylene glycol (PEG).

10. The lipid carrier of claim 9 wherein the PEG has a molecular weight of from about 500 to about 1500 Daltons and the carrier comprises greater than about 15 mol % PEG-lipid.

11. The lipid carrier of claim 10 wherein the PEG has a molecular weight of about 750 to about 1250 Daltons.

12. The lipid carrier of claim 10 wherein the PEG has a molecular weight of about 750 Daltons.

13. The lipid carrier of any one of claims 10-12, wherein the carrier comprises from about 15 to about 20 mol % PEG-lipid.

14. The lipid carrier of claim 13 wherein the PEG has a molecular weight of about 750 Daltons.

15. The lipid carrier of claim 9 wherein the PEG has a molecular weight of from about 1500 to about 5000 Daltons.

16. The lipid carrier of claim 15 wherein the molecular weight is from about 1500 to about 3000 Daltons.

17. The lipid carrier of claim 15 wherein the molecular weight is about 2000 Daltons.

18. The lipid carrier of any one of claims 15-17, wherein the carrier comprises at least about 12 mol % PEG-lipid.

19. The lipid carrier of any one of claims 15-17, wherein the carrier comprises at least about 15 mol % PEG-lipid.

20. The lipid carrier of claim 18 or 19, wherein the carrier comprises less than about 20 mol % PEG-lipid.

21. A lipid carrier according to any one of claims 1-20 wherein an amount of the carrier that would remain in a bloodstream of a warm blooded animal 4 hours after intravenous administration of the carrier is a value that is at least about 5 fold over the amount that would remain in the bloodstream of a reference.

22. The lipid carrier of claim 21 wherein the value is at least about 15 fold.

23. The lipid carrier of claim 21 wherein the value is at least about 25 fold.

24. The lipid carrier of claim 21 wherein the value is at least about 50 fold.

25. The lipid carrier of claim 21 wherein the value is at least about 75 fold.

26. The lipid carrier of claim 21 wherein the value is at least about 100 fold.

27. The lipid carrier of any one of claims 21-26 wherein the reference comprises 5 mol % or less of a hydrophilic polymer-lipid conjugate relative to total lipid content of the reference.

28. The lipid carrier of any one of claims 21-26 wherein the reference does not contain a hydrophilic polymer-lipid conjugate.

29. The lipid carrier of any one of claims 1-28 in which a non-lipid therapeutic agent is encapsulated.

30. The use of a lipid carrier comprising PS according to any one of claims 1-29 as a cytotoxic agent.

31. The use of a lipid carrier comprising PS according to of any one of claims 1-29 in the preparation of a medicament for treatment of cancer.

32. The use of a lipid carrier comprising PS according to any one of claims 1-29 in the preparation of a medicament for inducing thrombogenesis.
